(19) United States
(12) Patent Application Publication
(10) Pub. No.: US 2013/0012470 A1
(43) Pub. Date: Jan. 10, 2013

(54) ANTIVIRAL COMPOSITIONS AND METHODS OF THEIR USE

(75) Inventors: John Schlafer Colman, Margate, FL (US); Luis Ramon Romero, Doral, FL (US)

(73) Assignee: Humanitas Technology, LLC, Doral, FL (US)

(21) Appl. No.: 13/178,051

(22) Filed: Jul. 7, 2011

(51) Int. Cl. A61K 31/522; A61J 1/00; A61P 31/14; A61K 31/715; A61K 31/357; A61P 31/12

(52) U.S. Cl. 514/54; 514/450; 514/263.31; 206/570

(57) ABSTRACT

Novel compositions comprising viral fusion inhibition compounds and viral replication inhibition compounds as well as methods of their use are disclosed. The novel compositions are useful, inter alia, in the prevention, inhibition and/or treatment of Dengue Fever (DF) or Dengue Hemorrhagic Fever (DHF).

### FIELD OF THE INVENTION

The present invention relates to compositions and methods of using such compositions, inter alia, in the prevention, inhibition and/or treatment of Dengue Fever (DF) or Dengue Hemorrhagic Fever (DHF). More particularly, this invention relates to compositions comprising artesunate and at least one of a select group of anti-viral compounds, and their use, inter alia, as antiviral agents.

### BACKGROUND OF THE INVENTION

Dengue fever is the most important mosquito-borne viral disease affecting humans; its global distribution is comparable to that of malaria. An estimated 3.5 billion people live in areas at risk for endemic transmission. Each year, tens of millions of cases of Dengue Fever and hundreds of thousands of cases of Dengue Hemorrhagic Fever (DHF) occur, with the majority identified in tropical Asia, Latin America and the Caribbean. Wang et al., “A Small-Molecule Dengue Virus Entry Inhibitor,” Antimicrobial Agents and Chemotherapy, (53)5, 1823-1831 (2009). The fatality rate of DHF in most countries is about 5% of cases identified, with most fatal cases occurring in children and young adults, but the overall fatality rate should be capable of reduction to less than 1% with better treatment protocols. In comparison to the general population, the dengue fever fatality rate among pregnant women and young children is considerably higher, and may reach as high as 50% if the DF goes untreated.

The etiological agents reportedly involved are four serotypes of dengue virus (dengue virus serotype 1 [DENV-1], [DENV-2], [DENV-3], and [DENV-4]), which belong to the genus Flavivirus in the family Flaviviridae. Id. Infection by dengue virus is indicated as being initiated by fusion between the viral membrane and the host membrane. The fusion process is reported as being mediated by the dengue virus E protein in a pH-dependent manner. Stiasny, K., and Heinz, F. X., “Flavivirus Membrane Fusion,” J. Gen. Virol., 2006, (87) 2755-2766.

Subsequent attacks of dengue fever on a previously infected patient are another factor that tends to increase mortality rate where such repeated attacks occur. Each attack of dengue fever increases the likelihood and extent of red blood cell hemolysis, and makes the person more vulnerable to the next dengue fever infection.

While most dengue outbreaks occur in more tropical climates of the third world, there is a growing risk for dengue fever outbreaks in the continental United States. Two competent mosquito vectors, Ae. aegypti and Aedes albopictus, are present in areas of the U.S. Under certain circumstances, each is capable of transmitting dengue viruses. Mosquito-borne transmission has been detected six times in the last 25 years in south Texas (1980-2004) and has been associated with dengue epidemics in northern Mexico by Aedes aegypti and in Hawaii (2001-02) by Ae. albopictus. Moreover, numerous viruses are transported annually by travelers returning from tropical areas where dengue viruses are endemic. From 1977 to 2004, a total of 3,806 suspected cases of imported dengue were reported in the United States. Although some specimens collected were not adequate for laboratory diagnosis, 864 (23%) cases were confirmed as dengue. Many more cases probably go unreported each year because surveillance in the United States is passive. It relies on physicians' abilities to recognize the disease symptoms, inquire about the patient's travel history, obtain proper diagnostic samples, and report the cases to the proper governmental authorities. Filed reports of detected DF cases suggest that states in the southern and southeastern United States, where Ae. aegypti is found, are at risk for dengue transmission and sporadic outbreaks. Although travel-associated dengue and limited outbreaks do occur in the continental United States, the majority of reported U.S. contracted cases occur by endemic transmission in residents in some of the US territories. To monitor these endemic transmissions among US citizens, the U.S. Center for Disease Control (CDC) conducts laboratory-based passive surveillance with local governmental agencies. For example, the CDC collaborates in Puerto Rico with the Puerto Rico Department of Health.

The reasons for the dramatic global emergence of DF/DHF as a major public health problem are complex and not well understood. However, several important factors stand out as problematic. Major global demographic changes including uncontrolled urbanization coupled with concurrent population growth have resulted in substandard housing and inadequate water, sewer, and waste management systems, all of which increase Ae. aegypti population densities and facilitate transmission of Ae. Aegypti-borne disease.

In addition, the public health infrastructure in most countries has deteriorated. Competing priorities for limited financial and human resources have resulted in a “crisis mentality” having as an emphasis implementation of so-called emergency control methods in response to epidemics rather than on developing programs to prevent epidemic transmission. This restrictive approach has been particularly detrimental to dengue control. It leads to a reliance on passive surveillance by local doctors to detect increased transmission rates who often do not consider dengue in their differential diagnoses.

With passive dengue fever surveillance, it also becomes more likely that a dengue epidemic will reach or pass its peak before it is recognized and actions are taken to control its outbreak, which also factors in the non-local spread of the virus. Research has shown that air travel can provide an ideal mechanism for infected human transport of dengue viruses between population centers of the tropics, resulting in a frequent exchange of dengue viruses and other pathogens. With increasing air travel and passive DF outbreak surveillance, dengue translocation risks are heightened.

To further complicate the efforts to combat dengue fever, effective mosquito control is virtually nonexistent in most dengue-endemic countries. Considerable emphasis in the past has been placed on ultra-low-volume insecticide space sprays for adult mosquito control, a relatively ineffective approach for controlling Ae. aegypti. And while attenuated candidate vaccine viruses have been recently developed, no dengue vaccine is presently available, and efficacy trials of the attenuated viruses in human volunteers have not yet been initiated. Given the current level of advances in dengue vaccine research and development, it is unlikely that an effective dengue vaccine will be available for public use in the next 5 to 10 years.

Research directed to identification of compounds with anti-viral activity is advancing in view of a better general understanding of certain viruses, their transmission, infection, and replication within their hosts. For example, Reading et al. (U.S. Pat. No. 7,547,687) reports the use of certain androstene or androstane derivatives in methods of treating a wide range of viruses, including for example, Dengue virus types 1, 2, 3, and 4.

Nunes et al. (US Published Application Ser. No. 2011/0028385 A1) discloses certain compounds and methods said to be useful in the treatment of certain facultative or strict infections caused by intracellular microorganisms, wherein the compounds comprise certain immunomodulators and at least one anti-pathogenic agent. Among anti-pathogenic agents with antiprotozoal activity, Nunes identifies artemisinin and derivatives as natural extracts of Artemisia annua or synthetic derivatives thereof.

Johansen et al. (US Published Application Ser. No. 2008/0161324 A1) discloses certain compositions, methods, and kits useful in the treatment of viral diseases caused by, inter alia, a flaviviridae virus. Certain screening methods for identification of novel compounds that may be used to treat a viral disease are also reported.

Clinical studies have shown that carrageenans are active against common cold viruses. Marinomed Biotechnologie, an Austrian company, reported that a nasal spray containing carrageenan was effective as a treatment against the viral cause of the common cold. (Eccles R. et al., “Efficacy and Safety of an Antiviral Iota-Carrageenan Nasal Spray: a Randomized, Double-blind, Placebo-controlled Exploratory Study in Volunteers with Early Symptoms of the Common Cold,” Respiratory Research 2010, 11:108; also Grassauer et al., “Iota-Carrageenan is a Potent Inhibitor of Rhinovirus Infection,” Virology Journal 2008, 5:107 (PMID: 18817582 [PubMed—indexed for MEDLINE] PMCID: PMC2562995).

Talarico et al. reports the differential inhibition of dengue virus [DENV-2] infection in mammalian and mosquito cells by iota-carrageenan, as well as several virus assays. Talarico et al., J. Gen. Virol., June 2011 92:1332-1342, electronically pre-published on Feb. 16, 2011.

Grassauer et al. (US Published Application Ser. No. 2008/0131451 A1) discloses the use of carrageenan or mixtures thereof for the manufacture of certain antiviral pharmaceutical compositions for the treatment of rhinovirus infections. Other compositions reported by Grassauer et al. are disclosed as useful for the treatment of inflammation, allergies, and respiratory viruses. See US Published Application Ser. Nos. 2009/0298792 A1; 2010/0040658 A1; 2011/0091583 A1; and 2011/0059919 A1.

The bioactivity of artemisinin and its semi-synthetic derivative, artesunate, reportedly includes the inhibition of certain viruses, such as human cytomegalovirus and other members of the Herpesviridae family (e.g., herpes simplex virus type 1 and Epstein-Barr virus), hepatitis B virus, hepatitis C virus, and bovine viral diarrhea virus. See Efferth, et al., “The Antiviral Activities of Artemisinin and Artesunate”, Clin. Infect. Dis. (2008) 47 (6), 804-811; see also Sas et al., U.S. Pat. No. 7,842,719 disclosing artemisinin in the treatment of hepatitis C viral infections. Artesunate has certain reported antiviral properties in vitro and in in vivo human clinical trials. Milbradt, J. et al., “Sensitivity of human herpesvirus 6 and other human herpes viruses to the broad-spectrum antiinfective drug artesunate,” J Clin Virol., 2009 46(1):24-28.

Stiasny reported the identification of a number of small molecule Dengue virus inhibitors and noted that they may serve as molecular probes for the study of flavivirus entry into host cells. Stiasny, K., and Heinz, F. X., “Flavivirus Membrane Fusion,” J. Gen. Virol., 2006, (87) 2755-2766. Colman reports the use of combinations of berberine and artemisinin and its derivatives to treat malaria, diarrhea, travellers' diarrhea, dysentery, dengue fever, parasites cholera, and viruses. Colman et al., U.S. patent application Ser. No. 12/428,465, filed Apr. 22, 2009.

However, at present, prospects for reversing the recent trend of increased epidemic activity and geographic expansion of dengue are not promising. New dengue virus strains and serotypes will likely continue to be introduced into many areas where the population densities of Ae. aegypti are at high levels. The increase in dengue fever reported cases in Indonesia has doubled from 100,000 cases to 200,000 cases in 2007, suggesting that dengue fever cases are increasing almost exponentially.

Inasmuch as dengue fever in any of its various forms is a virulent and deadly illness with no generally accepted cure whose adverse impact on living species, including humans, is well documented, there continues to be a need for specific and effective remedies. Given the increased risk of dengue fever transmission, especially in third world countries where mosquito control is generally ineffective and sanitation conditions in many instances are poor, methods administering compounds or mixtures of compounds that not only target the viral fusion event or viral replication event of the dengue fever virion, but may in certain instances target both viral events, are desirable for preventing, inhibiting or treating dengue fever.

Thus, there is still an unfulfilled need for compounds and/or compositions that may be used in methods to attack the viral fusion/replication cycle, particularly where certain compounds may selectively target the viral fusion event while other compounds target the viral replication event to ameliorate, prevent, inhibit, reduce the severity thereof and or treat, inter alia, dengue fever in a host cell or patient having such host cells. The present invention is directed to these, as well as other important ends.

### SUMMARY OF THE INVENTION

Accordingly, the present invention is directed, in part, to pharmaceutical compositions for the prevention, inhibition, and/or treatment of dengue fever in any of its various forms or combinations of forms thereof. Preferably, the present invention is directed, in part, to compositions, comprising an effective amount of a viral fusion inhibitor compound or a pharmaceutically acceptable salt thereof; and an effective amount of a viral replication inhibitor compound or a pharmaceutically acceptable salt thereof.

In other embodiments, the present invention is directed to methods of preventing, inhibiting or treating dengue fever in a host cell comprising the step of administering to the host cell (or host cell in a patient in need thereof) an effective amount of a composition comprising an effective amount of a viral fusion inhibitor compound or a pharmaceutically acceptable salt thereof; and an effective amount of a viral replication inhibitor compound or a pharmaceutically acceptable salt thereof.

In certain embodiments, the present invention is directed to kits, comprising a container having a composition, said composition comprising an effective amount of a viral fusion inhibitor compound or a pharmaceutically acceptable salt thereof, and an effective amount of a viral replication inhibitor compound or a pharmaceutically acceptable salt thereof; and instructions for administering the oral dosage formulation.

In certain other embodiments, the present invention is directed to oral dosage compositions comprising an effective amount of a viral fusion inhibitor compound or a pharmaceutically acceptable salt thereof; and an effective amount of a viral replication inhibitor compound or a pharmaceutically acceptable salt thereof.

In other embodiments, the present invention is directed to methods of treating dengue shock syndrome in a patient in need thereof, comprising the step of administering to the patient an effective amount of a composition comprising an effective amount of a viral replication inhibitor compound or a pharmaceutically acceptable salt thereof; and an effective amount of an innate immune system modulator compound or a pharmaceutically acceptable salt thereof.

### DETAILED DESCRIPTION OF ILLUSTRATIVE EMBODIMENTS

As employed above and throughout the disclosure, the following terms, unless otherwise indicated, shall be understood to have the following meanings.

The term “virion” as used herein refers to a virus or virus particle comprising genetic material or “genes” made from either DNA or RNA; a protein coat that protects these genes; and, in some cases, an envelope of lipids that surrounds the protein coat.

The term “viral fusion” as used herein refers to the binding of the virus to specific molecules on the surface of a host cell. This specificity restricts the virus to a very limited type of cell because its surface protein can only react with certain other molecules on the host cell's surface. This mechanism has evolved to favor those viruses that only infect cells in which they are capable of reproducing.

The term “viral replication” as used herein refers to the stage where a cell uses viral messenger RNA in its protein synthesis systems to produce viral proteins. The RNA or DNA synthesis machinery of the cell produce the virus's DNA or RNA. This aspect of replication is followed by assembly and release of the virion. Assembly takes place in the cell when the newly created viral proteins and nucleic acid combine to form hundreds of new virus particles. Release occurs when the new viruses escape or are released from the cell.

The term “viral fusion inhibitor compound” as used herein refers to a compound that is capable of adversely affecting, interfering with or otherwise inhibiting, at least in part, at least one aspect of viral fusion to a host cell.

The term “viral replication inhibitor compound” as used herein refers to a compound that is capable of adversely affecting, interfering with or otherwise inhibiting, at least in part, at least one aspect of viral replication within and/or release from a host cell.

Assays that may be used to identify compounds that inhibit viral fusion, and in particular, those that inhibit the process of dengue fever virion fusion to a host cell, and/or those that may be used to identify compounds that inhibit viral replication, and in particular, those that inhibit the process of dengue fever virion replication within and/or release from a host cell, are disclosed in numerous publications, including, for example, Wang et al. and Shum et al. Wang et al., “A Small-Molecule Dengue Virus Entry Inhibitor,” Antimicrobial Agents and Chemotherapy, (53)5, 1823-1831 (2009); Shum et al., “High Content Assay to Identify Inhibitors of Dengue Virus Infection,” Assay and Drug Development Technologies, 8(5), 2010, 553-570.

The term “artesunate” as used herein refers to the succinic acid half ester derivative of dihydroartemisinin. Dihydroartemisinin may be obtained by sodium borohydride reduction of artemisinin, an unusual sesquiterpene lactone containing an epidioxide function. See Scheme 1. Artemisinin, or (ginghaosu), a clinically useful antimalarial agent was originally isolated from the plant Artemisia annua.

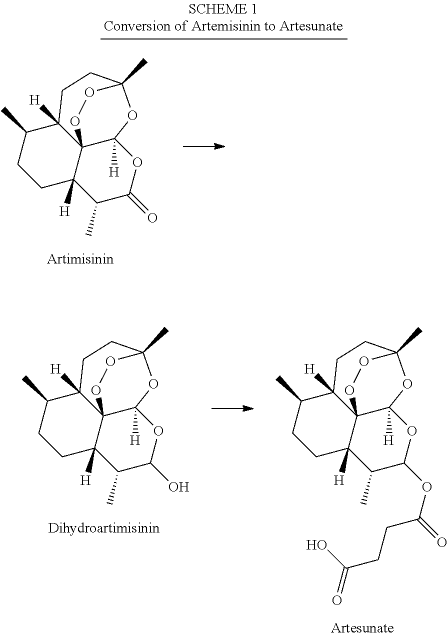

The present invention contemplates individual stereoisomers and/or combinations or mixtures of one or more stereoisomers and/or partial stereoisomers, as well as their mixtures. For example, artesunate and other derivatives of dihydroartemisinin have eight stereocenters, denoted by the asterisks in the illustration below (Ia).

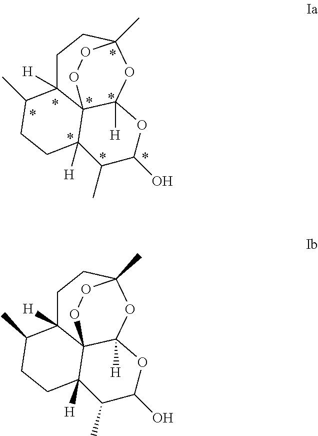

Each of the stereocenters of artesunate, artemisinin, or other derivatives of dihydroartemisinin may have an R or S configuration. Thus, Ia encompasses 28 (or 256) possible stereoisomers. In certain preferred embodiments, the artesunate is derived from the naturally occurring ketone artemisinin, whose stereochemistry has been reported. Accordingly, the stereochemistry of artesunate and/or dihydroartemisinin will mirror the stereochemistry of the seven stereochemical centers present in naturally occurring artemisinin in certain preferred embodiments of the invention (see Ib, above). Likewise, salts or other derivatives of artesunate and/or dihydroartemisinin may also have stereoisomeric structures with similar stereochemical assignments. Moreover, in certain preferred embodiments it is advantageous for the artesunate and/or dihydroartemisinin, or salts or other derivatives thereof to have a particular stereochemical configuration with regard to the lactol hydroxyl group represented by an asterisk in structure Ic (below), obtained by reduction of artemisinin. Accordingly, in certain preferred embodiments the configuration of the lactol-derived stereocenter is (R). In alternatively preferred embodiments, the configuration is (S).

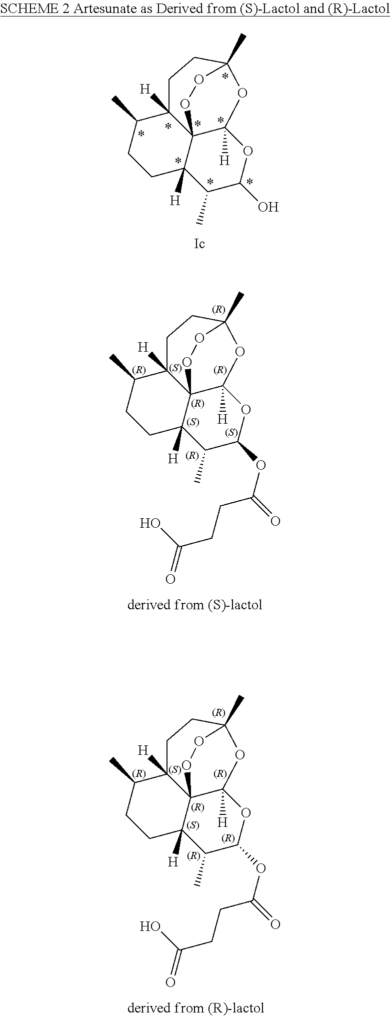

As used herein, the term “carrageenan” refers to a family of linear sulfated polysaccharides that are extracted from red seaweeds such as Rhodophyceae. The family of carrageenans includes, for example, the commercially available kappa, iota, and lambda carrageenans, among others. The carrageenans are typically high-molecular-weight polysaccharides made up of repeating galactose units and 3,6 anhydrogalactose (3,6-AG), wherein each repeating unit may be individually sulfated or non-sulfated in its nature. The units are joined by alternating alpha 1-3 and beta 1-4 glycosidic linkages. There are three main commercial classes of carrageenan: kappa, iota and lambda. The primary differences that influence the properties of kappa, iota, and lambda carrageenan are the number and position of the ester sulfate groups on the repeating galactose units.

Most carrageenan is now extracted from Kappaphycus alvarezii (formerly Eucheuma cottonii, and commercially was and is called “cottonii”) and Eucheuma denticulatum (formerly Eucheuma spinosum and commercially was and is called “spinosum”). The original source of carrageenan was Chondrus crispus (also known as irish moss gelose), and this is still used to a limited extent. Betaphycus gelatinum (formerly Eucheuma gelatinae) is used for a particular type of carrageenan. Some South American species that have previously been used to a limited extent are now gaining favor with carrageenan producers as they look for more diversification in the species available to them and the types of carrageenan that can be extracted. Gigartina skottsbergii, Sarcothalia crispate (formerly Iridaea ciliate) and Mazzaella laminaroides (formerly Iridaea laminaroides) are currently the most valuable species, all collected from natural resources in Chile. Small quantities of Gigartina canaliculata are harvested in Mexico. Hypnea musciformis has been used in Brazil.

“Pharmaceutically acceptable” refers to those compounds, materials, compositions, salts and/or dosage forms which, within the scope of sound medical judgment, are suitable for administration to patients without excessive toxicity, irritation, allergic response, or other problems or complications commensurate with a reasonable benefit/risk ratio.

“Salts” refer to derivatives of the disclosed compounds wherein the parent compound is modified by making acid or base salts thereof, or wherein the parent compound is in its zwitterionic form. When contacted with an acid, for example, resulting in the protonation of an amine functionality, the compound becomes associated with an anion, i.e., the counterion of the acid. When contacted with a base, for example, resulting in the deprotonation of an acid functionality, the compound is associated with a cation, i.e., the counterion of the base. Examples of salts include, but are not limited to, mineral or organic acid salts of basic residues such as amines, alkali or organic base salts of acidic residues such as carboxylic acids, and the like. Suitable mineral or organic acids or bases that may be employed in preparing salts of the compounds of the invention would be readily apparent to one of ordinary skill in the art, once placed in possession of the present application.

In certain preferred embodiments, the salts are “pharmaceutically acceptable salts,” which include, for example, conventional salts derived from pharmaceutically acceptable acids or bases, as well as internal or zwitterionic salts. Such pharmaceutically acceptable salts include those derived from inorganic acids such as hydrochloric, hydrobromic, sulfuric, sulfamic, phosphoric or nitric acid and the like; and salts prepared from organic acids such as acetic, propionic, succinic, glycolic, stearic, lactic, malic, tartaric, citric, ascorbic, pamoic, maleic, hydroxymaleic, phenylacetic, aspartic, glutamic, benzoic, salicylic, sulfanilic, acetoxybenzoic, fumaric, toluenesulfonic, naphthyldisulfonic, methanesulfonic, ethane disulfonic, oxalic or isethionic acid, and the like. Pharmaceutically acceptable salts also include those derived from metal bases, including alkali metal bases, for example, alkali hydroxides such as sodium hydroxide, potassium hydroxide and lithium hydroxide in which the metal is a monovalent species, alkaline earth metal bases, for example, alkaline earth metal hydroxides such as magnesium hydroxide and calcium hydroxide in which the metal is a polyvalent species, basic amines such as, for example, N,N′-dibenzylethylenediamine, arginine, chloroprocaine, choline, diethanolamine, ethylenediamine, meglumine (N-methylglucamine) and procaine, ammonium bases or alkoxides.

Physiologically acceptable salts as described herein may be prepared by methods known in the art, for example, by dissolving the free amine bases with an excess of the acid in aqueous alcohol, or neutralizing a free carboxylic acid with a metal base, preferably an alkali metal base such as a hydroxide, a substituted or unsubstituted ammonium hydroxide, an alkoxide, or an amine. In addition, it is well known to ordinarily skilled artisans that in compounds containing, for example, both a basic nitrogen atom and an acidic group, the nitrogen atom and the acidic functionalities may exist in equilibrium with their zwitterionic form depending, for example, on the characteristics of the involved aqueous medium including, for example, its ionic strength, pH, temperature, salts involved when the aqueous medium is in the form of a buffer, and the like. These zwitterionic salts are, in essence, internal pharmaceutically acceptable salts, and are contemplated to be within the scope of the present invention.

The term “ammonium base”, as used herein, refers to ammonium hydroxide (NH₄OH), as well as substituted ammonium hydroxides, i.e., NR₄OH, where one, two, three or four of the R groups may be, independently, alkyl, cycloalkyl, alkenyl, aryl, aralkyl, heteroaryl, or heterocycloalkyl. Exemplary substituted ammonium hydroxides include, for example, tetraalkyl ammonium hydroxides, such as tetramethyl ammonium hydroxide.

The term “alkoxide”, as used herein, refers to the product from the reaction of an alkyl alcohol with a metal. Exemplary alkoxides include, for example, sodium ethoxide, potassium ethoxide and sodium t-butoxide.

As used herein, the term “β-Glucan” (or beta-glucan) refers to polysaccharides of D-glucose monomers linked by β-glycosidic bonds. β-glucans are a diverse group of molecules that can vary with respect to molecular. mass, solubility, viscosity, and three-dimensional configuration. They occur most commonly as cellulose in plants, the bran of cereal grains, the cell wall of baker's yeast, certain fungi, mushrooms and bacteria. Yeast and medicinal mushroom derived β-glucans are notable for their ability to modulate the immune system. Research has shown that insoluble (1,3/1,6) β-glucan has greater biological activity than that of its soluble (1,3/1,4) β-glucan counterparts. Ooi, V. E. et al., “Immunomodulation and anti-cancer activity of polysaccharide-protein complexes”. Curr. Med. Chem. (2000) 7 (7): 715-29. The differences between β-glucan linkages and chemical structure are reportedly significant in regards to solubility, mode of action, and overall biological activity. Goodridge et al., “Activation of the innate immune receptor Dectin-1 upon formation of a ‘phagocytic synapse’,” Nature, 472, 471-475 (2011). According to Goodridge, Dectin-1 (also known as CLEC7A) is a pattern-recognition receptor expressed by myeloid phagocytes (macrophages, dendritic cells and neutrophils) that detects β-glucans in fungal cell walls and triggers direct cellular antimicrobial activity, including phagocytosis and production of reactive oxygen species (ROS).

Compounds described herein may be used or prepared in alternate forms. For example, many amino-containing compounds can be used or prepared as acid addition salts. Often such salts improve isolation and handling properties of the compound. The acid employed in forming acid addition salts is not generally limited. Pharmaceutically acceptable and pharmaceutically unacceptable acids may be used to prepare acid addition salts. For example, depending on the reagents, reaction conditions and the like, compounds as described herein can be used or prepared, for example, as their hydrochloride or tosylate salts. Similarly, compounds as described herein can be used or prepared, for example, as their oxalic acid or succinic acid salts, wherein one or both, preferably one, of the carboxylic acid groups in oxalic or succinic acid protonates the basic nitrogen atom that may be present in a viral fusion inhibitor compound or viral replication inhibitor compound of the invention.

Generally speaking, pharmaceutically unacceptable salts are not useful as medicaments in vivo. However, such salts may in certain cases demonstrate improved crystallinity and thus may be useful, for example, in the synthesis or physical testing of viral fusion inhibitor compounds or viral replication inhibitor, such as in connection with the formation, isolation and/or purification of viral fusion inhibitor compounds or viral replication inhibitor compounds and/or intermediates thereto. This may result, for example, in improved synthesis, purification or formulation by preparing and/or using compounds of the invention as salts that may not typically be considered to be pharmaceutically acceptable salts. These non-pharmaceutically acceptable salts may be prepared from acids or bases that are not typically considered to be pharmaceutically acceptable. Examples of such salts include, for example, acid addition salts prepared from trifluoroacetic acid, perchloric acid and tetrafluoroboric acid. Non-pharmaceutically acceptable salts may be employed in certain embodiments of the present invention including, for example, methods for the in vitro inhibition of viral fusion and/or replication by viral fusion inhibitor compounds or viral replication inhibitor compounds. In addition, if desired, such non-pharmaceutically acceptable salts may be converted to pharmaceutically acceptable salts by using techniques well known to the ordinarily skilled artisan, for example, by exchange of the acid that is non-pharmaceutically acceptable, for example, trifluoroacetic, perchloric or tetrafluoroboric acid, with an acid that is pharmaceutically acceptable, for example, the pharmaceutically acceptable acids described above.

Acid addition salts of the present invention include, for example, about one or more equivalents of monovalent acid per mole of the compound of the invention, depending in part on the nature of the acid as well as the number of basic lone pairs of electrons available for protonation. Similarly, acid addition salts of the present invention include, for example, about one-half or more equivalents of a divalent acid (such as, for example, oxalic acid or succinic acid) or about one third or more equivalents of trivalent acid (such as, for example, citric acid) per mole of the compound of the invention, depending in part on the nature of the acid as well as the number of basic lone pairs of electrons available for protonation. Generally speaking, the number of acid equivalents may vary up to about the number of equivalents of basic lone pairs of electrons in the compounds described herein.

Other examples of salts of the present invention which are derived from metal bases or basic amines include, for example, about one or more equivalents of monovalent metal or amine per mole of the compound of the invention, depending in part on the nature of the base as well as the number of available acidic protons. Similarly, salts of the present invention include, for example, about one-half or more equivalents of a divalent base (such as, for example, magnesium hydroxide or calcium hydroxide). Generally speaking, the number of basic equivalents may vary up to about the number of equivalents of acidic protons in the compounds described herein.

Salts of the present invention which are derived from metal bases or basic amines include, for example, about one or more equivalents of monovalent metal or amine per mole of the compound of the invention, depending in part on the nature of the base as well as the number of available acidic protons. Similarly, salts of the present invention include, for example, about one-half or more equivalents of a divalent base (such as, for example, magnesium hydroxide or calcium hydroxide). Generally speaking, the number of basic equivalents may vary up to about the number of equivalents of acidic protons in the compounds described herein. Non-pharmaceutically acceptable amines or metal bases may be employed in certain embodiments of the present invention including, for example, methods for the in vitro inhibition of viral fusion and/or replication by viral fusion inhibitor compounds or viral replication inhibitor compounds. In addition, if desired, such non-pharmaceutically acceptable salts may be converted to pharmaceutically acceptable salts by using techniques well known to the ordinarily skilled artisan, for example, by exchange of the metal cation or ammonium cation (derived from any applicable amine bases) that is non-pharmaceutically acceptable, for example, with a metal cation or ammonium cation that is pharmaceutically acceptable, for example, a metal cation, including monovalent metal cations such as a sodium, potassium or lithium cation, with sodium and lithium cations being preferred, and sodium cations being more preferred. In alternate embodiments, the metal cation may be a polyvalent cation, for example, a divalent cation such as a magnesium or calcium cation. In still other alternate embodiments, the cation may be, for example, an ammonium ion derived from a pharmaceutically acceptable amine base.

“Effective amount” refers to an amount of a compound as described herein that may be therapeutically effective to prevent, inhibit, reduce the severity of or treat the symptoms of one or more forms of dengue fever virus. Such forms of dengue fever virus include, but are not limited to, those pathological conditions associated with the administration of viral fusion inhibitor compounds or viral replication inhibitor compounds, wherein the treatment comprises, for example, affecting the fusion or replication of dengue fever virions by contacting cells, tissues or receptors with compounds and/or combinations of compounds of the present invention. Thus, for example, the term “effective amount,” when used in conjunction with viral fusion inhibitor compounds or viral replication inhibitor compounds for the prevention, inhibition, reduction in the severity of or treatment of the symptoms of one or more forms of dengue fever virus, refers to the prevention, inhibition, reduction in the severity of, or treatment of the viral condition. The term “effective amount,” when used in connection with other compounds independently or synergistically active against dengue fever virus fusion and replication, refers to the prevention, inhibition, reduction in the severity of, or treatment of one or more of the symptoms typically associated with one or more forms of dengue fever virus.

“In combination with,” “combination therapy,” and “combination products” refer, in certain embodiments, to the concurrent administration to a patient of one or more compounds or salts of the invention, in combination with one or more other compounds active in the prevention, inhibition, reduction in the severity of, or treatment of one or more of the symptoms of one or more forms of dengue fever virus.

The other optional compounds active in the prevention, inhibition, reduction in the severity of, or treatment of one or more of the symptoms of one or more forms of dengue fever virus may themselves further include one or more conventional components that may be designed to enhance the analgesic potency of the optional compound and/or reduce tolerance development to the optional compound, and/or other therapeutic agents described herein. When administered in combination, each component may be administered at the same time or sequentially in any order at different points in time. Thus, each component may be administered separately but sufficiently closely in time so as to provide the desired therapeutic effect.

“Dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by: (a) the unique characteristics of the active compound(s) and the particular therapeutic effect(s) to be achieved; and (b) the limitations inherent in the art of compounding such active compound(s).

“Patient” refers to animals, including mammals, preferably humans.

The terms “treat,” “treatment,” or “treating,” as used herein, generally refer to palliative (e.g., therapeutic), preventative (e.g., prophylactic), inhibitory, and/or curative treatment. Preferably, the terms “treat,” “treatment,” and/or “treating” refer to palliative, inhibitory, and/or curative treatment, with palliative and inhibitory treatment being more preferred. Even more preferably, the terms “treat,” “treatment,” or “treating” refer to palliative treatment.

The present invention is directed, in part, to viral fusion inhibitor compounds or salts thereof and/or viral replication inhibitor compounds or salts thereof, preferably compositions comprising viral fusion inhibitor compounds or salts thereof and/or viral replication inhibitor compounds or salts thereof that may prevent, inhibit, reduce the severity of, or treat the symptoms of one or more forms of dengue fever virus, preferably by adversely affecting, interfering with or otherwise inhibiting, at least in part, at least one aspect of viral fusion to, viral replication within, or viral release from a host cell. Embodiments are provided in which the viral fusion inhibitor compound and viral replication inhibitor compound interact synergistically or do so when administered in combination with other optional components that preferably prevent, inhibit, reduce the severity of, or treat the symptoms of one or more forms of dengue fever virus by adversely affecting, interfering with or otherwise inhibiting, at least in part, at least one aspect of viral fusion to, viral replication within, or viral release from a host cell.

The compositions comprising a viral fusion inhibitor compound and a viral replication inhibitor compound, and/or salt(s) thereof of the present invention demonstrate a surprisingly and unexpectedly advantageous profile of biological activities relative to profiles of biological activities of prior art compounds. In this regard, due to their desirable viral fusion and/or viral replication inhibiting properties, compositions and/or salts thereof as described herein may be useful, for example, in methods preventing, inhibiting or treating viral fusion of a dengue fever virion to a host cell in need thereof. Accordingly, the present compositions and/or pharmaceutically acceptable salts thereof may be useful in preventing, inhibiting, reducing the severity of, or treating any of the various forms of dengue fever virus. In preferred embodiments, the present compositions and pharmaceutically acceptable salts thereof may be employed in methods for the prevention, inhibition, reduction in the severity of, or treatment of dengue fever by adversely affecting, interfering with or otherwise inhibiting, at least in part, at least one aspect of viral fusion to, viral replication within, or viral release from a host cell. Such host cells may be treated in vitro or in vivo.

Compositions of the present invention may be potent and selective inhibitors of dengue fever virion fusion to, viral replication within, or viral release from a host cell, and/or may have highly desirable potencies as inhibitor compounds. In addition, compositions of the present invention, as well as any optional active components that may be co-administered with the compositions of the present invention, may demonstrate highly beneficial increases in in vivo oral bioavailability resulting in more predictable systemic exposure, and reduced variability in their pharmacokinetic behavior as compared to prior art compounds. This highly desirable profile of biological activities and pharmacokinetic properties in compounds of the present invention as compared to prior art compounds is surprising and unexpected.

Accordingly, in one embodiment, the present invention provides compositions, comprising an effective amount of a viral fusion inhibitor compound or a pharmaceutically acceptable salt thereof; and an effective amount of a viral replication inhibitor compound or a pharmaceutically acceptable salt thereof.

In certain preferred embodiments, the structures of the viral fusion inhibitor compound and the viral replication inhibitor compound differ with respect to each other.

The compositions and methods employing the compositions of the present invention comprise one or more viral fusion inhibitor compounds. In accordance with embodiments of the present invention such as, for example, compositions, a pharmaceutically active agent included therein may be a viral fusion inhibitor compound, such as piperazine or carrageenan, and more preferably carrageenan or a pharmaceutically acceptable salt thereof, or various combinations of the viral fusion inhibitor compound and/or one or more pharmaceutically acceptable salts thereof.

A wide variety of carrageenans are available which may be suitable for use in such methods and compositions. Carrageenans may act as either viral fusion inhibitors or viral replication inhibitors within the context of the present invention. Generally speaking, it is only necessary that the carrageenan assist in providing desired effect (for example, viral fusion inhibition or viral replication inhibition), and be capable of being incorporated into the present compositions and/or methods (discussed in detail below).

In some preferred embodiments of the invention, carrageenan, more preferably lambda, kappa, or iota or mixture thereof, still more preferably lambda or iota-carrageenan or mixture thereof, and yet more preferably lambda carrageenan, is provided in the compositions of the invention as a viral fusion inhibitor. In certain alternatively preferred embodiments, it is provided as a viral replication inhibitor.

Other examples of viral fusion inhibitor compounds include, for example, highly sulfated polysaccharides from fucoidan or algae; calcikum spirulan, nostoflan, or extract of Scoparia dulcis, or antiviral diterpene components contained therein, such as scoparic acid A, scoparic acid B, scoparic acid C, scopodiol, scopadulin, scopadulcic acid A (SDA), scopadulcic acid B (SDB), and/or scopadulcic acid C (SDC). Structures of these exemplary components shown below.

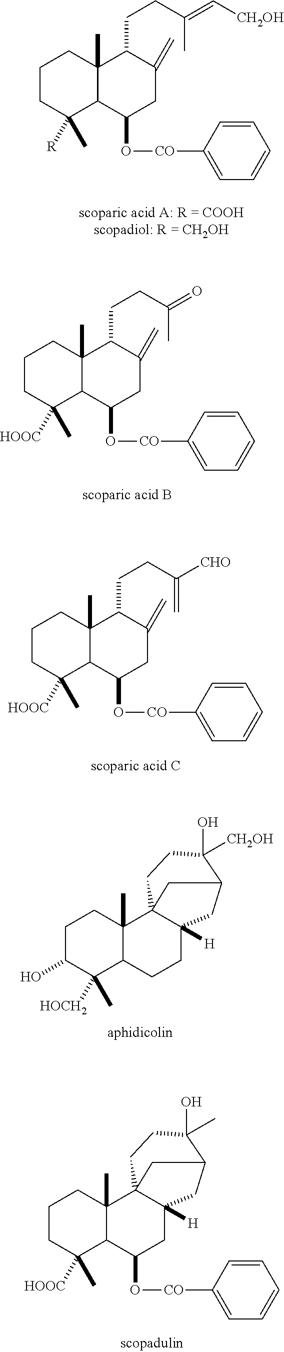

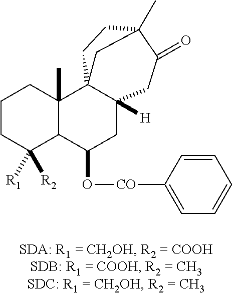

Still other examples of viral fusion inhibitors are disclosed in Table 1 of Wang, the disclosure of which is hereby incorporated herein by reference in its entirety. Wang et al., “A Small-Molecule Dengue Virus Entry Inhibitor,” Antimicrobial Agents and Chemotherapy, (53)5, 1823-1831 (2009). In certain preferred embodiments, the viral fusion inhibitor compound or salt thereof is selected from one of the compounds identified in Table 1 of Wang. Id., page 1826.

The compositions of the present invention and methods employing the compositions also comprise one or more viral replication inhibitor compounds. In some preferred embodiments, the viral replication inhibitor compound is selected from the group comprising artesunate, piperazine, carrageenan, acyclovir, gangcyclovir, or oseltamivir, or salt or combination of compound(s) and/or salt(s) thereof. In certain more preferred embodiments, the viral replication inhibitor compound or salt thereof is selected from the group comprising artesunate, carrageenan, or a combination thereof, and yet more preferably artesunate. Recognizing that dihydroartemisinin may also be active as a viral replication inhibitor compound against the various forms of dengue, and that artesunate or like derivative of dihydroartemisinin may be hydrolyzed at physiological pH or metabolized by the host to which it is administered, the invention contemplates artesunate or other derivatives of dihydroartemisinin, dihydroartemisinin, and mixtures thereof as alternately preferred viral replication inhibitor compounds in the compositions and/or methods of the present invention.

Also in accordance with embodiments of the present invention such as, for example, compositions, a pharmaceutically active agent included therein may be a viral replication inhibitor compound, such as artesunate, carrageenan, acyclovir, gangcyclovir, or oseltamivir, or combination thereof, or a pharmaceutically acceptable salt thereof, or various combinations of the viral replication inhibitor compound and/or one or more pharmaceutically acceptable salts thereof.

Viral fusion and/or viral replication inhibitor compounds of the invention, such as any of the compounds disclosed herein and/or identifiable by any of the assays noted herein, and salts thereof, also include other forms, such as their stereoisomers (except where specifically indicated), prodrugs, or any isomorphic crystalline forms thereof.

Compounds employed in the methods and compositions of the present invention may exist in prodrug form. As used herein, “prodrug” is intended to include any covalently bonded carriers which release the active parent drug, for example, the viral fusion inhibitor compound or viral replication inhibitor compound, or other formulas or compounds employed in the present methods and compositions in vivo when such prodrug is administered to a mammalian subject. The term “prodrug” also includes compounds which may be specifically designed to maximize the amount of active species that reaches the desired site of reaction and which themselves may be inactive or minimally active for the activity desired, but through biotransformation are converted into biologically active metabolites. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example a viral fusion inhibitor compound or viral replication inhibitor compound, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

The compounds of the present invention may be prepared and or isolated from natural sources in a number of ways well known to those skilled in the art. The compounds can be synthesized, for example, by known methods, or variations thereon as appreciated by the skilled artisan. All processes disclosed in association with the present invention are contemplated to be practiced on any scale, including milligram, gram, multigram, kilogram, multikilogram or commercial industrial scale.

While not intending to be bound by any theory or theories of operation, it is contemplated that inabilities to prevent, inhibit, or treat dengue fever may result from failure to attack both viral fusion and viral replication. Thus, the use of separate compounds or combinations of compounds (or pharmaceutically acceptable salts thereof) wherein each compound or combination targets viral fusion or replication in such a way that both events are targeted by a composition of the present invention may effectively prevent, inhibit, or treat dengue fever. According to one aspect of the present invention, administration of a composition of the invention may block or interrupt dengue virion fusion with a host cell and/or dengue virion replication within or release from a host cell. In vivo or in vitro administration to a host cell is contemplated to be within the scope of the present invention, allowing, for example, patient administration on the one hand and screening of compounds and/or compositions against modified or newly discovered strains of dengue fever on the other.

In accordance with certain embodiments of the present invention, there are provided methods for administering to a patient a composition of the invention comprising an effective amount of a viral fusion inhibitor compound or a pharmaceutically acceptable salt thereof; and an effective amount of a viral replication inhibitor compound or a pharmaceutically acceptable salt thereof.

In addition, the compositions may further include one or more compounds that may be designed to enhance the anti-viral potency of the inhibitor compounds and/or to reduce anti-viral tolerance development. The optional components would be readily apparent to one of ordinary skill in the art, once apprised of the teachings of the present disclosure.

Another embodiment of the invention provides compositions for use in methods for inhibiting viral fusion or viral replication of a dengue fever virion, said composition comprising a pharmaceutically acceptable carrier and an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

The methods of the present invention may be useful in preventing, inhibiting or treating viral fusion of a dengue fever virion to a host cell, viral replication of a dengue fever virion within a host cell, or viral release from a host cell. Accordingly, administration of the present compositions and/or pharmaceutically acceptable salts thereof may be useful in preventing, inhibiting, reducing the severity of, or treating any of the various forms of dengue fever virus.

In certain preferred embodiments, it may be advantageous to administer the viral fusion inhibitor compound, at least in part, via a second route of administration. In other words, some of the viral fusion inhibitor compound is administered to a patient via a first chosen route of administration while the remaining portion of the viral fusion inhibitor compound is administered to the patient via a second chosen route of administration. Alternatively, the method may comprise administration of a first viral fusion inhibitor compound by one route of administration and a second viral fusion inhibitor compound via an alternative route of administration. Employing such methods may assist administration by enhancing bioavailability or absorption of the viral fusion inhibitor compound or second viral fusion inhibitor compound. In certain preferred embodiments, the compositions of the present invention may be co-administered with a second amount of a carrageenan compound or mixture of carrageenan compounds thereof, more preferably lambda, kappa, or iota or mixture thereof, still more preferably lambda or iota-carrageenan or mixture thereof, and yet more preferably lambda carrageenan.

In other preferred embodiments of the methods of the invention, one or more second viral fusion inhibitor compounds may be co-administered to a host cell or a patient in need thereof, wherein the one or more second viral fusion inhibitor compound(s) may be administered in form that is the same or different when compared to that of the first viral fusion inhibitor compound of the present invention. Alternately, the one or more second viral fusion inhibitor compound(s) may be administered by the same or by a different means relative to the first viral fusion inhibitor compound to a patient in need thereof. The structure of the second viral fusion inhibitor compound(s) and that of the one or more viral fusion inhibitor compound(s) comprising the compositions of the present invention may be the same or different.

In particular preferred embodiments, it is beneficial to administer, at least in part, a viral fusion inhibitor compound in the form of a mucosal spray, such as a nasal spray. The viral fusion inhibitor compound in the mucosal spray may be the same or different from the viral fusion inhibitor compound in the composition of the invention being co-administered. The spray may be administered before, during, or after the time that the composition of the invention is administered to a patient.

In certain more preferred embodiments, a carrageenan is provided, at least in part, in the form of the co-administered mucosal spray, preferably administered in combination with an oral form of a pharmaceutical composition of the present invention for the prevention, inhibition, reduction of the severity of, and/or treatment of dengue fever.

In some other preferred embodiments of the invention, the optional mucosal spray comprises a carrageenan or mixture of carrageenans, preferably lambda or iota carrageenan or mixture thereof, more preferably iota carrageenan in a sterile saline solution, preferably a sea salt solution, more preferably a Dead Sea salt solution. The mucosal spray may also contain at least one component selected from the group consisting of Lysozyme enzyme, zinc gluconate, and an antibacterial spray preservative. In certain preferred embodiments, the mucosal spray contains from about 0.1 to about 0.9% by weight carrageenan based on the total weight of the mucosal spray; more preferably from about 0.2 to about 0.6%, with about 0.25 to about 0.5% being even more preferred and all combinations and subcombinations thereof. In a typical example, a nasal spray may comprise about 1.2 grams/L of carrageenan, about 5 grams sodium chloride/L, and about 20 ml of water.

In yet other preferred embodiments of the invention, the optional mucosal spray further comprises a beta-glucan. beta-Glucan is an immune system modulator compound that may prime the innate immune system to protect the body. Certain beta-glucans may be obtained from Biothera, Inc., a private healthcare and pharmaceutical company located in Eagan, Minn. beta-Glucans from Biothera, Inc., and in particular, any of those that are capable of binding Dectin-1, are preferable in certain of the mucosal sprays and or methods of treatment of the present invention. Innate immune systems beta-glucans are described in numerous patents and publications, including, for example, U.S. Pat. Nos. 5,223,491; 5,519,009; 5,397,773; 5,702,719; 5,705,184; 6,369,216; 6,630,310; 7,022,685; 7,566,704; and 7,786,094; the disclosure of each of which is hereby incorporated herein by reference, in its entirety.

Yet another embodiment of the invention provides methods for preventing, inhibiting, reducing the severity of, and/or treating dengue fever comprising administering to a patient an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

In other embodiments, the present invention is directed to methods of preventing, inhibiting, reducing the severity of, or treating viral fusion of a dengue fever virion to a host cell in need thereof comprising the step of administering to the host cell an effective amount of a composition comprising an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

In still other embodiments, the present invention is directed to methods of preventing, inhibiting, reducing the severity of, or treating viral fusion of a dengue fever virion to a host cell in a patient comprising the step of administering to the patient an effective amount of a composition comprising an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

In some other embodiments, the present invention is directed to methods of preventing, inhibiting, reducing the severity of, or treating viral replication of a dengue fever virion in a host cell in need thereof comprising the step of administering to the host cell an effective amount of a composition comprising an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

In still other embodiments, the present invention is directed to methods of preventing, inhibiting or treating viral replication of a dengue fever virion in a host cell in a patient comprising the step of administering to the patient an effective amount of a composition comprising: an effective amount of a viral fusion inhibitor compound or a pharmaceutically acceptable salt thereof; and an effective amount of a viral replication inhibitor compound or a pharmaceutically acceptable salt thereof; preferably wherein the structures of the viral fusion inhibitor compound and the viral replication inhibitor compound differ with respect to each other.

One of the most severe complications resulting from a dengue fever virus infection is the risk of a patient suffering from Dengue Shock Syndrome, or DSS. The present invention contemplates the administration of compositions of the present invention for the treatment of Dengue Shock Syndrome in a patient in need thereof.

Thus, in certain embodiments, the present invention is directed to methods of treating dengue shock syndrome in a patient comprising the step of administering to the patient an effective amount of a composition comprising an effective amount of a viral replication inhibitor compound or a pharmaceutically acceptable salt thereof; and an effective amount of an innate immune system modulator compound or a pharmaceutically acceptable salt thereof.

Preferably, the innate immune system modulator compound is a beta-glucan, more preferably a beta-1,3- or beta-1,6-glucan, still more preferably a beta-1,3-glucan.

In certain preferred embodiments directed to treatment of DSS, administration is initially carried out parenterally. Injections of viral replication inhibitor compound (about 50 to 100 mg, preferably about 60 mg to about 90 mg, more preferably about 75 mg to 85 mg of viral replication inhibitor compound) on a per injection basis are typically performed several times per day, preferably about every six hours. The innate immune system modulator compound is likewise administered to the patient. The viral replication inhibitor compound and the innate immune system modulator compound may be administered together or separately. The injections are typically continued until the patient is out of coma. Once a patient has come out of coma, an oral dosage regimen may replace parenteral administration of each of the active compounds. For example, the patient may be orally administered artesunate tablets (100 mg) three times daily for a period of about four more days.

In certain embodiments, the present invention is directed to kits, comprising a container having an oral dosage composition comprising an effective amount of a viral fusion inhibitor compound or a pharmaceutically acceptable salt thereof, and an effective amount of a viral replication inhibitor compound or a pharmaceutically acceptable salt thereof; and instructions for administering the oral dosage composition.

In certain other embodiments, the present invention is directed to oral dosage compositions comprising an effective amount of a viral fusion inhibitor compound or a pharmaceutically acceptable salt thereof; and an effective amount of a viral replication inhibitor compound or a pharmaceutically acceptable salt thereof.

In certain preferred embodiments of the kits or dosage forms of the present invention, the structures of the viral fusion inhibitor compound and the viral replication inhibitor compound differ with respect to each other.

The inhibitor compounds may be administered alone or may be combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety. The relative proportions of active ingredient and carrier may be determined, for example, by the solubility and chemical nature of the compounds, chosen route of administration, and standard pharmaceutical practice.

Compounds as described herein may be administered to a mammalian host in a variety of forms adapted to the chosen route of administration, e.g., orally or parenterally. Parenteral administration in this respect includes administration by the following routes: intravenous; intramuscular; subcutaneous; intraocular; intrasynovial; transepithelial including transdermal, ophthalmic, sublingual and buccal; topically, including ophthalmic, dermal, ocular, and rectal; and nasal inhalation via insufflations and aerosols.

The dosage of the compounds of the invention may vary depending upon various factors such as, for example, the pharmacodynamic characteristics of the particular agent and its mode and route of administration, the age, health and weight of the recipient, the nature and extent of the symptoms, the kind of concurrent treatment, the frequency of treatment, and the effect desired. Generally, small dosages may be used initially and, if necessary, increased by small increments until the desired effect under the circumstances is reached. Generally speaking, oral administration may require higher dosages.

Although the viral fusion inhibitor compound(s) and viral replication inhibitor compound(s) of the present invention may be administered as the pure chemicals, it is preferable to present the active ingredient(s) as a pharmaceutical composition. The invention thus further provides a pharmaceutical composition comprising one or more of the viral fusion inhibitor compound(s) and viral replication inhibitor compound(s) of the present invention, together with one or more pharmaceutically acceptable carriers, and, optionally, other therapeutic and/or prophylactic ingredients. The carrier(s) must be acceptable in the sense of being compatible with the other ingredients of the composition and not deleterious to the recipient thereof.

The viral fusion inhibitor compound(s) and viral replication inhibitor compound(s) and/or their compositions of the present invention may be administered in an effective amount by any of the conventional techniques well-established in the medical field. The viral fusion inhibitor compound(s) and viral replication inhibitor compound(s) of the present invention employed in the methods of the present invention may be administered by any means that results in the contact of the active agents with the agents' site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety.

Compounds of the present invention can be administered to a mammalian host in a variety of forms adapted to the chosen route of administration, e.g., orally or parenterally. Parenteral administration in this respect includes administration by the following routes: intravenous; intramuscular; subcutaneous; intraocular; intrasynovial; transepithelial including transdermal, ophthalmic, sublingual and buccal; topically, including ophthalmic, dermal, ocular, and rectal; and nasal inhalation via insufflation and aerosol.

The active compound(s) may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 20 to about 1000 mg of artesunate, more preferably from about 40 to about 100 mg per dose, and all combinations and subcombinations of ranges and specific amounts of active compound therein, taken from about one to about three times daily. Oral dosage ranges for dihydroartemisinin and other derivatives and/or analogs thereof may be prepared so that an oral dosage unit form contains from about 1 to about 1500 mg of dihydroartemisinin and/or other derivative and/or analog and all combinations and subcombinations of ranges and specific amounts of active compound therein, taken from about one to about three times daily.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique that yields a powder of the active ingredient, plus any additional desired ingredient from the previously sterile-filtered solution thereof.

The therapeutic compounds and/or compositions of the present invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds and/or compositions of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The combination products of this invention, such as pharmaceutical compositions comprising the viral fusion inhibitor compound(s) and viral replication inhibitor compound(s) and/or their compositions, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the viral fusion inhibitor compound(s) and viral replication inhibitor compound(s) and/or their compositions of the present invention may be administered at the same time (that is, together), or in any order. When not administered at the same time, preferably the administration of the viral fusion inhibitor compound(s) and viral replication inhibitor compound(s) and/or their compositions of the present invention occurs less than about one hour apart, more preferably less than about 30 minutes apart, even more preferably less than about 15 minutes apart, and still more preferably less than about 5 minutes apart. Preferably, administration of the combination products of the invention is oral, although other routes of administration, as described above, are contemplated to be within the scope of the present invention. Although it is preferable that the viral fusion inhibitor compound(s) and viral replication inhibitor compound(s) and/or their compositions of the present invention are both administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

In an alternately preferred embodiment, the compositions of the invention are provided along with a mucosal spray comprising a viral fusion inhibitor compound. The compositions of the present invention and the mucosal spray may be administered at the same time (that is, together), or in any order. When not administered at the same time, preferably the administration of the viral fusion inhibitor compound(s) and viral replication inhibitor compound(s) and/or their compositions of the present invention occurs less than about one hour apart, more preferably less than about 30 minutes apart, even more preferably less than about 15 minutes apart, and still more preferably less than about 5 minutes apart. Preferably, administration of the compositions of the invention is oral, especially when provided in combination with a mucosal spray comprising a viral fusion inhibitor compound to be co-administered, although other routes of administration for the compositions, as described above, are contemplated to be within the scope of the present invention. Although it is preferable that the viral fusion inhibitor compound(s) and viral replication inhibitor compound(s) and/or their compositions of the present invention are both administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired. In some preferred embodiments, the viral fusion inhibitor is administered in the form of a gargle or mouthwash solution.

Although the proper dosage of the combination products of this invention will be readily ascertainable by one skilled in the art, once armed with the present disclosure, by way of general guidance, where the viral fusion inhibitor compound(s) and viral replication inhibitor compound(s) and/or their compositions of the present invention, for example, typically a daily dosage may range from about 0.01 to about 100 milligrams of the viral fusion inhibitor compound(s) (and all combinations and subcombinations of ranges therein) and about 0.001 to about 100 milligrams of the viral replication inhibitor compound (and all combinations and subcombinations of ranges therein), per kilogram of patient body weight. Preferably, the a daily dosage may be about 0.01 to about 30 milligrams of the viral fusion inhibitor compound(s) and about 0.01 to about 30 milligrams of the viral replication inhibitor compound per kilogram of patient body weight. Even more preferably, the daily dosage may be from about 0.5 to about 10 milligrams of the viral fusion inhibitor compound(s) and from about 1 to about 10 milligrams of the viral replication inhibitor compound per kilogram of patient body weight. Yet more preferably, the daily dosage may be from about 0.5 to about 5 milligrams of the viral fusion inhibitor compound(s) and from about 1 to about 8 milligrams of the viral replication inhibitor compound per kilogram of patient body weight. Still more preferably, the daily dosage may be from about 0.5 to about 2 milligrams of the viral fusion inhibitor compound(s) and from about 3 to about 5 milligrams of the viral replication inhibitor compound per kilogram of patient body weight. With regard to a typical dosage form of this type of combination product, such as a tablet, the viral fusion inhibitor compound(s) generally may be present in an amount of about 15 to about 200 milligrams, and the viral replication inhibitor compound in an amount of about 15 to about 300 milligrams.

Particularly when provided as a single dosage form, the potential exists for a chemical interaction between the combined active ingredients (for example, the viral fusion inhibitor compound(s) and the viral fusion replication compound(s)). Alternatively, one or more of the viral fusion inhibitor compound, viral replication inhibitor compound or other actives may be capable of degradation in the gastrointestinal tract of a patient in advance of its assimilation by the body. For either of these reasons, the preferred dosage forms of the combination products of this invention are formulated such that although the active ingredients are combined in a single dosage form, the physical contact between the active ingredients (or between one or more of the active ingredients and the patient's gastrointestinal tract) is minimized (that is, reduced). In particular aspects of the present invention's compositions, a carrageenan is provided, at least in part, as a component in an oral formulation, preferably wherein the carrageenan is enterically coated to reduce physical contact between the carrageenan and the gastrointestinal tract.

In order to minimize contact, one embodiment of this invention where the product is orally administered provides for a combination product wherein one active ingredient is enteric coated. By enteric coating one or more of the active ingredients it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material that effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

Pharmaceutical kits useful in, for example, the treatment, inhibition or prevention of dengue fever, which comprise a therapeutically effective amount of viral fusion inhibitor compound(s) along with a therapeutically effective amount of viral replication inhibitor compound(s) of the invention, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The viral fusion inhibitor compound(s) and the viral replication inhibitor compound(s) may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit. In certain preferred embodiments, the kits further comprise a container comprising a carrageenan adapted for topical or mucosal use. In certain alternatively preferred embodiments, the kits further comprise a dosage form of a mosquito repellent compound, preferably wherein said mosquito repellent compound is vitamin B6, or an analog, derivative or pharmaceutically acceptable salt thereof and guidelines for administration of the mosquito repellent dosage form. Oral dosages provided in certain preferred kits of the invention are typical oral, such as tablets, capsules and the like. Dosages of vitamin B6 provided in such oral dosage forms for a typical adult male are typically within the range from about 1 milligram to about 1,000 milligrams; preferably from about 40 milligrams to 800 milligrams with from about 200 milligrams to about 400 milligrams being even more preferred. Instructions for such dosages are provided with kits containing the oral dosage form. Generally the vitamin B6 oral dosage form may be taken on a once or twice per day regimen.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The dose may also be provided by controlled release of the compound, by techniques well known to those in the art.

Compounds of the present invention may be used in methods to adversely affect, interfere with or otherwise inhibit, at least in part, at least one aspect of viral fusion to, viral replication within, or viral release from a host cell, particularly dengue fever virion fusion and/or replication/release events. Such adversely affecting, interfering with, or otherwise inhibiting, at least in part, at least one aspect of viral fusion to, viral replication within, or viral release from a host cell may be accomplished by contacting the host cell or dengue fever virion in vitro or in vivo with an effective amount of a composition of the invention. Preferably, the contacting step is conducted in an aqueous medium, preferably at physiologically relevant ionic strength, pH, and the like. In vitro methods of adversely affecting, interfering with or otherwise inhibiting, at least in part, at least one aspect of viral fusion to a host cell, or viral replication within or release from a host cell may involve, for example, pharmaceutically acceptable salts or non-pharmaceutically acceptable salts, and may be used, for example, to evaluate the prevention, inhibition, reduction in the severity of or treatment properties toward the viral condition of other compounds or compositions in assays in which the present compounds may be used as an assay standard, and the like.

When ranges are used herein for physical properties, such as weight percent, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Various modification of the invention, in addition to those described herein, will be apparent to those skilled in the art from the foregoing description. Such modifications are also intended to fall within the scope of the appended claims.

The invention illustratively disclosed herein suitably may be practiced in the absence of any element which is not specifically disclosed herein. The invention illustratively disclosed herein suitably may also be practiced in the absence of any element which is not specifically disclosed herein and that does not materially affect the basic and novel characteristics of the claimed invention.

The invention is contemplated to be practiced on any suitable scale.

### Embodiment 1

A composition, comprising:

an effective amount of a viral fusion inhibitor compound or a pharmaceutically acceptable salt thereof; and

an effective amount of a viral replication inhibitor compound or a pharmaceutically acceptable salt thereof;
wherein the structures of said viral fusion inhibitor compound and said viral replication inhibitor compound differ with respect to each other.

### Embodiment 2

A composition according to Embodiment 1, wherein the viral replication inhibitor compound comprises artesunate.

### Embodiment 3

A composition according to Embodiment 1, wherein the viral replication inhibitor compound is selected from the group consisting of acyclovir, gangcyclovir, or oseltamivir, or combination thereof.

### Embodiment 4

A composition according to Embodiment 1, wherein the viral replication inhibitor compound comprises a carrageenan.

### Embodiment 5

A composition according to any one of Embodiments 1 to 4, wherein the viral fusion inhibitor compound comprises a carrageenan.

### Embodiment 6

A composition according to any one of Embodiments 1 to 5, further comprising a pharmaceutically acceptable carrier.

### Embodiment 7

A method of preventing, inhibiting or treating dengue fever in a host cell, said method comprising the step of:

administering to said host cell an effective amount of a composition according to any one of Embodiments 1 to 6.

### Embodiment 8

A method of preventing, inhibiting or treating dengue fever in a patient in need thereof, said method comprising the step of:

administering to said patient an effective amount of a composition according to any one of Embodiments 1 to 6.

### Embodiment 9

A method according to Embodiment 8, wherein said viral fusion inhibitor compound is adapted for topical or mucosal administration.

### Embodiment 10

A method according to Embodiment 8 or 9, wherein said viral fusion inhibitor compound is adapted for administration as a nasal spray.

### Embodiment 11

A method according to Embodiment 8, wherein said viral fusion inhibitor compound is adapted for ocular administration.

### Embodiment 12

A method according to Embodiment 8, viral fusion inhibitor compound is adapted for administration as a gargle solution.

### Embodiment 13

A kit, comprising:

a container having a composition,

wherein said composition comprising any one of Embodiments 1 to 6; and instructions for administering said composition.

### Embodiment 14

A kit according to Embodiment 15, wherein said viral fusion inhibitor compound is adapted for topical or mucosal administration.

### Embodiment 15

A kit according to Embodiment 15 or 16, wherein said viral fusion inhibitor compound is adapted for administration as a nasal spray.

### Embodiment 16

A kit according to Embodiment 15, wherein said viral fusion inhibitor compound is adapted for ocular administration.

### Embodiment 17

A kit according to Embodiment 16, wherein said viral fusion inhibitor compound is adapted for administration as a gargle solution.

### Embodiment 18

A method of treating dengue shock syndrome in a patient in need thereof, said method comprising the step of:

administering to said patient an effective amount of a composition comprising:

an effective amount of a viral replication inhibitor compound or a pharmaceutically acceptable salt thereof; and

an effective amount of an innate immune system modulator compound or a pharmaceutically acceptable salt thereof.

### Embodiment 19

A method according to Embodiment 18, wherein said composition is administered parenterally or orally.

### Embodiment 20

A method according to Embodiment 18 or 19, wherein said composition is administered parenterally.

### Embodiment 21

A method according to Embodiment 18 or 19, wherein said composition is administered orally.

### Embodiment 22

A method according to any one of Embodiments 18 to 21, wherein the viral replication inhibitor compound comprises artesunate.

### Embodiment 23

A method according to any one of Embodiments 18 to 22, wherein the innate immune system modulator compound comprises a beta (1,3)-glucan.

### Embodiment 24

A method according to any one of Embodiments 9 to 12, wherein said viral replication inhibitor compound comprises artesunate.

### Embodiment 25

A method according to any one of Embodiments 9 to 12, wherein said viral fusion inhibitor compound comprises a carrageenan.

### Embodiment 26

A kit according to any one of Embodiments 13 to 17, wherein said viral replication inhibitor compound comprises artesunate.

### Embodiment 27

A kit according to any one of Embodiments 13 to 17, wherein said viral fusion inhibitor compound comprises a carrageenan.

What is claimed:

1. A composition comprising:
an effective amount of a viral fusion inhibitor compound or a pharmaceutically acceptable salt thereof; and
an effective amount of a viral replication inhibitor compound or a pharmaceutically acceptable salt thereof;
wherein the structures of said viral fusion inhibitor compound and said viral replication inhibitor compound differ with respect to each other.

2. A composition according to claim 1, wherein said viral replication inhibitor compound comprises artesunate.

3. A composition according to claim 2, wherein said viral fusion inhibitor compound comprises a carrageenan.

4. A composition according to claim 1, wherein the viral fusion inhibitor compound comprises a carrageenan.

5. A composition according to claim 4, wherein the viral replication inhibitor compound is selected from the group consisting of acyclovir, gangcyclovir, and oseltamivir, or combination thereof.

6. A composition according to claim 4, wherein said viral replication inhibitor compound comprises a carrageenan.

7. A composition according to claim 1, wherein the viral replication inhibitor compound is selected from the group consisting of acyclovir, gangcyclovir, and oseltamivir, or combination thereof.

8. A composition according to claim 1, further comprising a pharmaceutically acceptable carrier.

9. A method of preventing, inhibiting or treating dengue fever in a host cell, said method comprising the step of:
administering to said host cell an effective amount of a composition comprising:
an effective amount of a viral fusion inhibitor compound or a pharmaceutically acceptable salt thereof; and
an effective amount of a viral replication inhibitor compound or a pharmaceutically acceptable salt thereof;
wherein the structures of said viral fusion inhibitor compound and said viral replication inhibitor compound differ with respect to each other.

10. A method according to claim 9, wherein said composition further comprises a pharmaceutically acceptable carrier.

11. A method according to claim 10, wherein said viral fusion inhibitor compound is adapted for topical or mucosal administration.

12. A method according to claim 11, wherein said viral fusion inhibitor compound is adapted for administration as a nasal spray.

13. A method according to claim 11, wherein said viral fusion inhibitor compound is adapted for ocular administration.

14. The method according to claim 11, wherein said viral fusion inhibitor compound is adapted for administration as a gargle solution.

15. A kit comprising:
a container having a composition,
wherein said composition comprises:
an effective amount of a viral fusion inhibitor compound or a pharmaceutically acceptable salt thereof, and
an effective amount of a viral replication inhibitor compound or a pharmaceutically acceptable salt thereof,
wherein the structures of said viral fusion inhibitor compound and said viral replication inhibitor compound differ with respect to each other; and
instructions for administering said composition.

16. A kit according to claim 15, wherein said viral fusion inhibitor compound is adapted for topical or mucosal administration.

17. A kit according to claim 16, wherein said viral fusion inhibitor compound is adapted for administration as a nasal spray.

18. A kit according to claim 15, wherein said viral fusion inhibitor compound is adapted for ocular administration.

19. A kit according to claim 15, wherein said viral fusion inhibitor compound is adapted for administration as a gargle solution.

20. A method of treating dengue shock syndrome in a patient in need thereof, said method comprising the step of:
administering to said patient an effective amount of a composition comprising:
an effective amount of a viral replication inhibitor compound or a pharmaceutically acceptable salt thereof; and
an effective amount of an innate immune system modulator compound or a pharmaceutically acceptable salt thereof.

21. A method according to claim 20, wherein said composition is administered parenterally or orally.

22. A method according to claim 20, wherein said composition is administered parenterally.

23. The method according to claim 20, wherein said composition is administered orally.

24. A method according to claim 20, wherein said viral replication inhibitor compound comprises artesunate.

25. A method according to claim 20, wherein said innate immune system modulator compound comprises a beta (1,3)-glucan.
